(19) United States
(12) Patent Application Publication
(10) Pub. No.: US 2022/0093295 A1
(43) Pub. Date: Mar. 24, 2022

(54) MAGNETIC BEADS, METHOD OF MAKING AND METHOD OF USE THEREOF

(71) Applicant: Nanophase Technologies Corporation, Romeoville, IL (US)

(72) Inventors: Harry W. Sarkas, Shorewood, IL (US); Kevin Cureton, Evanston, IL (US)

(21) Appl. No.: 17/490,092

(22) Filed: Sep. 30, 2021

Related U.S. Application Data
(63) Continuation of application No. PCT/US2021/050269, filed on Sep. 14, 2021.
(60) Provisional application No. 63/079,875, filed on Sep. 17, 2020.

(51) Int. Cl. H01F 1/03; C12Q 1/686; C01G 49/06; C01G 49/00; C01G 49/08

(52) U.S. Cl. CPC H01F 1/0315; C12Q 1/686; C01G 49/06; B82Y 5/00; C01G 49/0072; C01G 49/08; C01G 49/0063

(57) ABSTRACT

Magnetic beads comprise a plurality of magnetic nanoparticles, dispersed in a non-magnetic matrix. The magnetic beads have an average particle size of 0.1 μm to 100 μm. The matrix may comprise an inorganic metal oxide or a polymer. The magnetic beads have a specific surface area of at least 40 m²/g.

### BACKGROUND

Magnetic beads have been used to separate nucleic acids from complex biological mixture, for further analysis (such as sequencing), processing or modification. Such beads typically have a surface presenting chemical groups which have an affinity for nucleic acids, such as —OH groups. Preferably, the beads are large enough for easy separation from the complex biological mixture using a magnet, while being small enough to remain suspended in the mixture to bind the nucleic acids of interest. A optimal particle size is 1 μm to 2 μm. Larger beads are more easily separated, but they tend to settle out of the mixture quickly and have a low surface area to volume. Smaller beads have a high surface area to volume, but are slow to separate from the mixture under the action of a magnetic field. Furthermore, it is desirable to maximize the magnetic moment of the beads, as the stronger the attraction of the beads to the magnet used to separate the beads from the mixture, the more quickly the beads can be collected and the more of the beads, and therefore more of the nucleic acids, that are collected.

Magnetic beads have been prepared by starting with magnetic nanoparticles, that is particles having a particle size of at most 1 μm (1000 nm). Magnetic moment is the product of volume and saturation magnetization due to internal alignment of atomic spins. Saturation magnetization is maximized by minimizing the number of magnetic domains (that is, regions of aligned atomic spins) within each magnetic nanoparticle. Single domain superparamagnetic nanoparticles may also be used. Depending on the material, the maximum size for single domain magnetic nanoparticles is typically much less than 100 nm.¹

There has been a trade-off between controlling the size of the beads, the shape of the beads, the selection of magnetic nanoparticles within the beads, and the amount of magnetic material in the beads. For example, in EP Patent No. 757,106, substantially spherical core-shell magnetic nanoparticles having a size of 0.2 μm to 0.4 μm are coated with a porous silica using tetraethoxysilane, producing substantially spherical magnetic beads have a porous silica coating and diameter of 0.5 μm to 15 μm, and having a magnetic metal oxide content of about 10% to 60%.² The size of the beads is controlled by the size of the magnetic nanoparticles in a tradeoff with the magnetic metal oxide content, and the shape of the magnetic beads is controlled by the shape of the magnetic nanoparticles. The magnetic nanoparticles are required to be larger than single domain magnetic nanoparticles, or the magnetic beads would be too small or have too little magnetic metal oxide content.

Magnetic glass beads have been formed by dispersing very small magnetic particles, such as single domain magnetic nanoparticles, into glass, such as described in U.S. Pat. Pub. No. 2005/0266462.³ A sol-gel process is used to disperse the magnetic nanoparticles in a gel matrix containing silica (SiO₂) and other constituents of glass including B₂O₃ and Al₂O₃, together with alkali metals such as sodium (Na). The mixture is then sprayed to form particles of the desired size and shape, which are then carefully sintered below the melting point to form the desired magnetic glass beads. The inclusion of alkali metals and other components of soft glass such as B₂O₃ is necessary to keep the sintering temperature below a temperature which would cause a loss of the magnetic properties. This process is complex due to the need to control the viscosity of the sol-gel so that it can be sprayed, and slow because of the rate at which 1 μm to 2 μm particles can be formed by spraying. The sintering process causes a loss of surface area, as the sintering process fills in pores and leads to a more uniform spherical shape. Table 4 of U.S. Pat. Pub. No. 2005/0266462 describes the BET surface area of several compositions, and the highest surface area achieved was 26.85 m²/g.

In another process magnetic beads are formed from a dispersion of superparamagnetic nanoparticle using an emulsion as a template, followed by free-radical polymerization.⁴ The magnetite nanoparticles, having a diameter of about 3 nm to 7 nm, were made hydrophobic by coating with oleic acid. A ferrofluid emulsion of the nanoparticles in hexane was formed which included benzophenone as a polymerization initiator, and SDS as the surfactant. To control the size of the oil-phase droplets, the emulsion was forced through a membrane having pores of the desired size (2 μm or 5 μm), and then the hexane evaporated. The SDS was then replaced with a polymerizable alcohol to provide —OH groups on the surface. The microparticles were formed by mixing the emulsion with acrylic acid and polymerizable alcohol, and then polymerized using ultraviolet light. The process is complex due to the number of steps involved.

### SUMMARY

In a first aspect, the present invention is magnetic beads, comprising: (i) a plurality of magnetic nanoparticles, dispersed in (ii) a non-magnetic inorganic oxide matrix. The magnetic beads have an average particle size of 0.1 μm to 100 μm, the magnetic nanoparticles have an average particle size of 20 nm to 50 nm, the non-magnetic inorganic oxide matrix contains neither Group I nor Group II elements, and does not contain boron, and the magnetic beads contain at least 75% by weight of the plurality of magnetic nanoparticles, and retain a saturation magnetization of at least 75% of the bulk saturation of the magnetic nanoparticles, based on the weight of the magnetic beads.

In a second aspect, the present invention is magnetic beads, comprising: (i) a plurality of magnetic nanoparticles, dispersed in (ii) a non-magnetic inorganic oxide matrix. The magnetic beads have an average particle size of 0.1 μm to 100 μm, the magnetic nanoparticles have an average particle size of 20 nm to 50 nm, the magnetic beads have a specific surface area of at least 40 m²/g, and the magnetic beads contain at least 75% by weight of the plurality of magnetic nanoparticles, and retain a saturation magnetization of at least 75% of the bulk saturation of the magnetic nanoparticles, based on the weight of the magnetic beads.

In a third aspect, the present invention is magnetic beads, comprising a plurality of magnetic nanoparticles, dispersed in a polymer matrix. The magnetic beads have an average particle size of 0.1 μm to 100 μm, and the polymer matrix does not contain moieties of a PEG functionalized surfactant.

In a fourth aspect, the present invention is a method of making magnetic beads, comprising forming a solid dispersion comprising magnetic nanoparticles dispersed in a matrix; and grinding the solid dispersion, to form magnetic beads having an average particle size of 0.1 μm to 100 μm.

### Definitions

The term “particle size” for means the average diameter of the image of the particle as viewed by electron microscopy or light microscopy. The term “particle size” is used in this manner unless otherwise stated. The term “average particle size” means the average of the particle sizes of a collection of particles (for particles having an average particle size of at least 500 nm) or that calculated using a spherical model from the specific surface area of particles measured in m²/g determined using the Brunauer-Emmett-Teller method (BET method) consistent with fully-dense particles (for particles have an average particle size of less than 500 nm), unless otherwise stated. The terms “powder”, “beads” and “particles” are used interchangeably.

The term “nanoparticle” means a particle have an average particle size of at most 1 μm (1000 nm).

The phrase “the bulk saturation magnetization of the magnetic nanoparticles” means the bulk saturation magnetization of the material of which the magnetic nanoparticles are formed, and does not mean the saturation magnetization of the magnetic nanoparticles.

All percentages (%) are weight/weight percentages, unless otherwise indicated.

All temperatures are reported to an accuracy of +/−5° C.

The specific surface area (SSA) of beads is measured in m²/g and is determined using the Brunauer-Emmett-Teller (BET) method.

### BRIEF DESCRIPTION OF THE DRAWINGS

FIG. 1 is a graph of the average yield percent as a percentage of the input DNA for various magnetic beads.

FIG. 2 is a graph of the average recovery by weight versus the weight of the input DNA for various magnetic beads.

FIG. 3 is a graph of the average yield percent as a percentage of the input RNA for various magnetic beads.

FIG. 4 is a graph of the average recovery by weight versus the weight of the input RNA for various magnetic beads.

FIG. 5 is a graph of the cycle number and the baseline subtracted fluorescence (RFU) for two magnetic beads with various input DNA concentrations.

FIG. 6 is a bar graph showing the Cq values of two magnetic beads at 1 ng and 10 ng of input DNA.

FIG. 7 is a graph of the cycle number and the baseline subtracted fluorescence (RFU) for two magnetic beads with various input RNA concentrations.

FIG. 8 is a bar graph showing the Cq values of two magnetic beads at 10 ng and 50 ng of input RNA.

### DETAILED DESCRIPTION

The prior processes for forming magnetic beads all use a bottom-up approach of constructing the beads from smaller materials, such as magnetic particles, liquids or emulsions, and controlling the size and composition of the beads as they are made. The present invention uses a different approach, forming a bulk material of the desired composition and then using grinding to control the size of the beads, in a top-down approach to forming magnetic beads. By separating the formation of the composition of the magnetic beads from the formation of beads themselves, the process can be both simplified and sped up.

The present invention includes forming a dispersion of magnetic nanoparticles, optionally single domain superparamagnetic nanoparticles, in a non-magnetic matrix, followed by grinding to form beads. The dispersion may be formed by surface polymerization, chemical deposition or melt processing. Optionally, the surface of the magnetic beads may be modified to improve the affinity for nucleic acids or other biological substance of interest. The present invention includes magnetic beads including a plurality of magnetic nanoparticles in a non-magnetic matrix.

The ability of magnetic beads to collect nucleic acid is proportional to the surface area of the beads, so a higher surface area provides better nucleic acid collection properties. In bottom-up approaches, sintering is required to form the bead and hold the bead together. The sintering process reduces the surface area of the beads because the melting causes the surface to become smoother and pores are filled. Preferably the magnetic beads of the present invention have a higher surface area to improve nucleic acid collection, compared to processes that include sintering. Glass beads that are sintered have a strong correlation between size and BET surface area because sintering causes a loss of porosity and reticulation, while the beads of the present application have a BET surface area that is nearly independent from size because the beads retain porosity and reticulation. The magnetic beads are solid, and the magnetic nanoparticles are preferably cross-linked together. The magnetic beads preferably have a specific surface area of at least 40 m²/g, such as 40 m²/g to 275 m²/g, including 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, and 270 m²/g, and ranges therebetween.

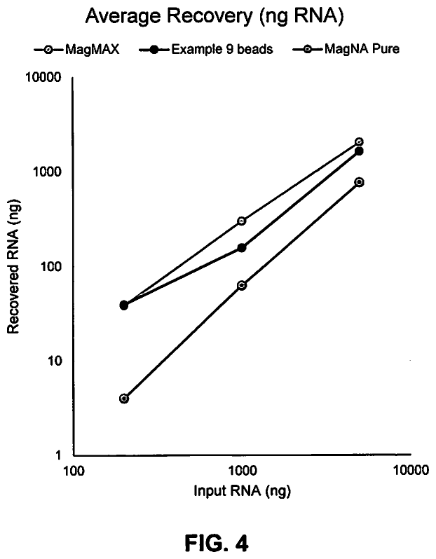

The magnetic nanoparticles preferably have an average particle size of at most 100 nm, such a 1 nm to 100 nm, preferably an average particle size of 10 nm to 70 nm, and most preferably 20 nm to 50 nm. Optionally, the magnetic nanoparticles are superparamagnetic. Preferably, the magnetic nanoparticles are single domain magnetic nanoparticles. The maximum particle size for a variety of superparamagnetic and single domain magnetic nanoparticles are described in Majetich et al, FIG. 4.¹

Preferably, the magnetic nanoparticles comprise gamma phase iron oxide and/or ferrite materials. Examples of ferrites included MₓO_{y}.Fe₂O₃, where M is at least one metal element, such as a Group 2, 3, 4, 5, 6, 7, 8, 9, 10, 11, 12, 13, 14 or 15 metal element, such as Ca, Sr and Ba (Group 2); Zn (Group 12); Fe, Co and Ni (Group 9); Mn (Group 7); Y and Lanthanide series elements such as La and Ce (Group 3); and Bi (Group 15), with x=1 to 4 and y=1 to 4. Examples include FeO.Fe₂O₃, (aka Fe₃O₄), ZnFe₂O₄, BiFeO₃, BaFeO₃, MnFe₂O₄, REO.Fe₂O₃ where REO=Y or a Lanthanide series element (such as Ce or La). Mixtures, doped materials and solid solutions may also be used.

The non-magnetic matrix may contain oxides, glasses, polymers, organic compounds and moieties, and mixtures thereof. Preferably, the non-magnetic matrix includes inorganic oxide such as SiO₂ (including Si(O—)₄ moieties), Al₂O₃ (including Al(O—)₃ moieties), TiO₂ (including Ti(O—)₄ moieties), and mixtures thereof. Preferably, Group 1 (such as Na and K) and Group 2 (such as Ca and Sr) elements are not present in the matrix. Preferably, boron (B) is not present in the matrix. Preferably, the matrix is not sintered or melted after addition of the magnetic nanoparticles.

The magnetic beads are formed from the dispersion, so both will have similar or the same composition. Preferably, the magnetic beads and/or the dispersion will contain at least 80%, more preferably at least 90% magnetic nanoparticles, including 90% to 98%, such as 91%, 92%, 93%, 94%, 95%, 96% and 97%.

The dispersion may be formed by coating the magnetic nanoparticles with the matrix material, such as by using chemical deposition or a modified chemical vapor deposition. Alternatively, the magnetic nanoparticles may be mixed with a polymerizable material, and then polymerization is used to form the matrix. Alternatively, the magnetic nanoparticles may be dispersed into a liquid polymer which then solidified, or a sol-gel which is then dried or solidified, to form the matrix. Grinding is then used to form the magnetic beads from the dispersion.

Preferably the magnetic beads have an average particle size of 0.1 μm to 100 μm, more preferably 0.2 μm to 10 μm, most preferably 0.5 μm to 5 μm, including 0.6 μm, 0.7 μm, 0.8 μm, 0.9 μm, 1.0 μm, 1.2 μm, 1.4 μm, 1.6 μm, 1.8 μm, 2.0 μm, 2.5 μm, 3.0 μm, 3.5 μm, 4.0 μm, 4.5 μm and values therebetween. The magnetic beads are formed by grinding the dispersion, so the average particle size may be easily controlled by the grinding process. Optionally, the magnetic beads may be sorted, for example using a sieve, to narrow the size distribution of the magnetic beads.

Preferably, the magnetic beads have a saturation magnetization of at least 75% of the bulk saturation magnetization of the magnetic nanoparticles present within the beads, more preferably at least 85%, and most preferably at least 90%. For example, the magnetic beads may have a saturation magnetization of 75% to 95%, including at least 76%, 77%, 78%, 79%, 80%, 81%, 82%, 83%, 84%, 85%, 86%, 87%, 88%, 89%, 90%, 91%, 92%, 93%, and 94%, of the bulk saturation magnetization of the magnetic nanoparticles present within the beads, and values therebetween.

Optional, the magnetic beads may be surface treated to enhance affinity for the desired biological compound. For example, poly(ethylene glycol) (PEG) may be conjugated to the surface to increase the amount of —OH groups. Alternatively, PEG may be conjugated to the surface, and then another agent, such as biotin or streptavidin may be conjugated to the PEG.

Magnetic beads may be used to isolate or purify nucleic acids. The purification or isolation of nucleic acids may be useful in a variety of biology methodologies, such as nucleic acid sequencing, direct detection of particular nucleic acid by nucleic acid hybridization and nucleic acid sequence amplification techniques such as polymerase chain reaction (PCR).

The method of isolating or purifying from a sample may include providing a sample in a solution containing nucleic acids such as RNA or DNA, reversibly binding the nucleic acid to magnetic beads, holding the magnetic beads in place using a magnet or a magnetic force, washing the solution to remove materials that are not bound to the magnetic beads such as proteins or debris, and eluting the nucleic acid from the magnetic beads using a buffer solution. In order to prepare the sample, lysing agents and neutralizing agents may be introduced to the sample in solution. The washing may be carried out 1, 2, 3, 4, 5 or more times to remove unwanted debris or other biological material from the magnetic beads and nucleic acids bound to the magnetic beads. Elution can be accomplished by heating the environment of the particles with bound nucleic acids and/or raising the pH of such environment. Agents which can be used to aid the elution of nucleic acid from paramagnetic particles include basic solutions such as potassium hydroxide, sodium hydroxide or any compound which will increase the pH of the environment to an extent sufficient that electronegative nucleic acid is displaced from the beads. An example of such a method is described in the MAGMAX™ Total Nucleic Acid Isolation Kit (THERMO FISHER SCIENTIFIC®, #AM1840).

The magnetic beads may be included in a kit. The kit may include buffers, such as lysis buffers, digestion buffers, rebinding and elution buffers. The kit may include wash solutions, processing plates, and elution plates. The kit may include lysing agents, such as protease K to disrupt the sample. Examples of kits include the MAGMAX™ Total Nucleic Acid Isolation Kit (THERMO FISHER SCIENTIFIC®, #AM1840), AMPure XP (BECKMAN COULTER®), RNAclean XP (BECKMAN COULTER®), MAGNESIL® (PROMEGA®), DYNABEADS™ (THERMO FISHER SCIENTIFIC®) and MagNA Pure 24 (ROCHE®).

### EXAMPLES

### Example 1: Synthesis of Single Domain Gamma Iron Oxide Magnetic Nanoparticles

Gamma-phase iron oxide (maghemite) particles were produced via the transferred arc methods described in U.S. Pat. Nos. 5,460,701 and 5,874,684 from high purity iron (>99.9% Fe) feedstock. The process was conducted using an Ar/H₂ mixture (65%/35%) as cathode gas, a specific power input of 34.4 kW/kg Fe₂O₃ and quench gas input of 8.4 ft³ air/kg Fe vapor where the quench air is introduced at the closest point to the origin projected merged plasma jet that maintains a stable arc. The average transport air flow was 20,000 ft³ air/kg Fe₂O₃. The resultant material had a specific surface area of 50 m²/g (BET method) corresponding to an equivalent average particle diameter of 23 nm. The phase purity of the product was examined using X-ray powder diffractometry and determined to be >98% gamma phase as evidenced by the absence of the main (104) peak of the alpha phase (hematite) at 33.28 degrees 2-theta.

### Example 2: Synthesis of Single Domain Gamma Iron Oxide Magnetic Nanoparticles

Gamma-phase iron oxide (maghemite) particles were produced via the free-burning arc methods described in U.S. patent application Ser. No. 10/172,848 and U.S. Pat. No. 7,517,513 from high purity FCC grade iron powder. The process was conducted using a specific power input of 22.3 kW/kg Fe₂O₃ and quench gas input of 4.4 ft³ air/kg Fe vapor where the quench air is introduced at the closest point to the origin projected merged plasma jet that maintains a stable arc. The average transport air flow was 4,125 ft³ air/kg Fe₂O₃. The resultant material had a specific surface area of 38 m²/g (BET method) corresponding to an equivalent average particle diameter of 30 nm. The phase purity of the product was examined using X-ray powder diffractometry and determined to be >98% gamma phase as evidenced by the absence of the main (104) peak of the alpha phase (hematite) at 33.28 degrees 2-theta. The saturation magnetization of the resulting product was measured using an alternating gradient magnetometer at 300K to be 73±1 emu/g. This represents 96% of the bulk value of 76 emu/g for maghemite.

### Example 3: Synthesis of Single Domain Zinc Ferrite Magnetic Nanoparticles

Zinc ferrite (ZnFe₂O₄) particles were produced via the process described in Example 2 by co-feeding high purity FCC grade iron powder and SHG grade Zn powder in a 2:1 molar ratio into the cathodic arc column. The resultant material had a specific surface area of 41 m²/g (BET method) corresponding to an equivalent average particle diameter of 28 nm. The crystal phase of the product was examined using X-ray powder diffractometry and determined to be face centered cubic.

### Example 4: Synthesis of Single Domain Bismuth Ferrite Magnetic Nanoparticles

Bismuth ferrite (BiFeO₃) particles were produced via the process described in Example 2 by co-feeding high purity FCC grade iron powder and Bi₂O₃ powder (99.9% purity) in a 1:4.17 mass ratio into the cathodic arc column. The resultant material had a specific surface area of 17 m²/g (BET Method) corresponding to an equivalent average particle diameter of 43 nm. The crystal phase of the product was examined using X-ray powder diffractometry and determined to have a rhombohedral distorted perovskite structure.

### Example 5: Synthesis of Single Domain Manganese Ferrite Magnetic Nanoparticles (Prophetic)

Manganese ferrite (MnFe₂O₄) particles were produced via the process described in Example 2 by co-feeding high purity FCC grade iron powder and MnO₂ powder in a 1:1.28 mass ratio into the cathodic arc column. The resultant material had an average particle diameter of 30 nm based on specific surface area measurement. The crystal phase of the product was examined using X-ray powder diffractometry and determined to have a cubic spinel structure.

### Example 6: Synthesis of Single Domain Complex Doped Ferrite Magnetic Nanoparticles (Prophetic)

Samarium doped zinc manganese ferrite particles were produced via the process described in Example 2 by co-feeding high purity FCC grade iron powder, MnO₂ powder, SHG grade Zn powder, and Sm₂O₃ powder in a 1.0:0.64:0.25:0.00057 mass ratio into the cathodic arc column. The resultant material had an average particle diameter of 30 nm based on specific surface area measurement.

### Example 7: Synthesis of Single Domain Fe₃O₄ Magnetic Nanoparticles

The powder of Example 1 was thermally reduced under a 5% H₂/95% N2 atmosphere at 525° C. to yield Fe₃O₄ powder. The resultant black material had a specific surface area of 26 m²/g (BET method) corresponding to an equivalent average particle diameter of 44 nm.

### Example 8: Formation of a Dispersion of Single Domain Gamma Iron Oxide Magnetic Nanoparticles in a Silica Matrix

1.93 kg of tetraethoxysilane (CAS Number 78-10-4) were added to 5.00 kg of the powder of Example 2 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol reaction product. The resulting powder was examined by thermogravimetric analysis (TGA) and found to have a loss on drying at 105° C. of less than 0.3% and be devoid of any ignitable components. The material was determined to have a composition of 9.9% SiO₂ and 90.1% Fe₂O₃ using X-ray fluorescence (XRF). The particle size distribution of the material was measured via static light scattering (ISO 13320:2009 Particle size analysis—Laser diffraction methods) using a Horiba® LA-960 Particle Size Analyzer. The material was observed to have an extremely broad particle size distribution with a mean particle diameter of 17.0 microns and an associated standard deviation of 26.1 microns for the distribution. The saturation magnetization of the resultant material can be computed to be 65.7 emu/g from the composition. This represents 86.4% of the value of the saturation magnetization value for the corresponding bulk magnetic material of the composite.

### Example 9: Preparation of Magnetic Beads from a Dispersion of Single Domain Gamma Iron Oxide Magnetic Nanoparticles in a Silica Matrix

A fraction of the powder of Example 8 was processed via comminution using a method exemplified in U.S. Pat. No. 3,614,000 to achieve a target mean diameter for the composite magnetic bead. An orbital jet mill having a diameter of 4 inches was used along with an injection pressure of 95 PSI and a grind chamber pressure of 95 PSI. Raw material powder was fed to the process at a rate of 5 kg/hour. The particle size distribution of the resultant magnetic bead material was then measured via static light scattering. The resultant magnetic bead material had a narrow Gaussian particle size distribution with a mean particle diameter of 1.14 microns and an associated standard deviation of 0.58 microns for the distribution. The specific surface area of the resultant magnetic bead material was measured using the BET method and found to be 69 m²/g.

### Example 10: Preparation of Magnetic Beads from a Dispersion of Single Domain Gamma Iron Oxide Magnetic Nanoparticles in a Silica Matrix

A fraction of the powder of Example 8 was processed via comminution using a method exemplified in U.S. Pat. No. 3,614,000 to achieve a target mean diameter for the composite magnetic bead. An orbital jet mill having a diameter of 4 inches was used along with an injection pressure of 72 PSI and a grind chamber pressure of 72 PSI. Raw material powder was fed to the process at a rate of 5 kg/hour. The particle size distribution of the resultant magnetic bead material was then measured via static light scattering. The resultant magnetic bead material had a narrow Gaussian particle size distribution with a mean particle diameter of 2.10 microns and an associated standard deviation of 1.13 microns for the distribution. The specific surface area of the resultant magnetic bead material was measured using the BET method and found to be 69 m²/g.

### Example 11: Formation of a Dispersion of Single Domain Gamma Iron Oxide Magnetic Nanoparticles in a Silica Matrix

4.33 kg of tetraethoxysilane (CAS Number 78-10-4) were added to 5.00 kg of the powder of Example 2 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol reaction product. The resulting powder was examined by thermogravimetric analysis (TGA) and found to have a loss on drying at 105° C. of less than 0.3% and be devoid of any ignitable components. The material was determined to have a composition of 20.0% SiO₂ and 80.0% Fe₂O₃ using X-ray fluorescence (XRF). The saturation magnetization of the resultant material can be computed to be 58.4 emu/g from the composition. This represents 76.8% of the value of the saturation magnetization value for the corresponding bulk magnetic material of the composite. This material may be converted to magnetic microbeads using the methods described in Example 9 and Example 10.

### Example 12 (Prophetic): Formation of a Dispersion of Single Domain Gamma Iron Oxide Magnetic Nanoparticles in an Alumina Matrix

2.23 kg of aluminum isopropoxide (CAS Number 555-31-7) were added to 5.00 kg of the powder of Example 2 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol reaction product. The resulting powder has a nominal composition of 10.0% Al₂O₃ and 90.0% Fe₂O₃ along with a corresponding saturation magnetization value of 65.7 emu/g from the composition. This material may be converted to magnetic microbeads using the methods described in Example 9 and Example 10.

### Example 13 (Prophetic): Formation of a Dispersion of Single Domain Gamma Iron Oxide Magnetic Nanoparticles in a Titania Matrix

1.98 kg of titanium isopropoxide (CAS Number 546-68-9) were added to 5.00 kg of the powder of Example 2 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol reaction product. The resulting powder has a nominal composition of 10.0% TiO₂ and 90.0% Fe₂O₃ along with a corresponding saturation magnetization value of 65.7 emu/g from the composition. This material may be converted to magnetic microbeads using the methods described in Example 9 and Example 10.

### Example 14 (Prophetic): Formation of a Dispersion of Single Domain Gamma Iron Oxide Magnetic Nanoparticles in an Aluminosilicate Matrix

1.40 kg of aluminum isopropoxide (CAS Number 555-31-7) and 0.71 kg of tetraethoxysilane (CAS Number 78-10-4) were each added to 5.00 kg of the powder of Example 2 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol reaction product. The resulting powder has a nominal composition of 10.0% amorphous aluminosilicate (Al₂SiO₅) and 90.0% Fe₂O₃ along with a corresponding saturation magnetization value of 65.7 emu/g from the composition. This material may be converted to magnetic microbeads using the methods described in Example 9 and Example 10.

### Example 15 (Prophetic): Formation of a Dispersion of Single Domain Gamma Iron Oxide Magnetic Nanoparticles in a Silica-PEG Matrix

0.193 kg of tetraethoxysilane (CAS Number 78-10-4) were added to 5.00 kg of the powder of Example 2 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol reaction product and the powder is subsequently brought to a temperature of 50° C. A sufficient quantity of a solution of polyethylene glycol (CAS Number 25322-68-3) in water (500 mg polyethylene glycol/ml) is sprayed onto the powder while under agitation to deliver 0.5 kg of polyethylene glycol. Under agitation the mixture is brough to a temperature of 110° C. under applied vacuum to remove the water fraction. The resultant dry powder is then brought to a temperature of 130° C. and held for 1 hour to graft the polyethylene glycol to the particle surfaces by the method described in U.S. Pat. No. 2,657,149 via reaction with the silanol groups from the first reaction step. The resulting material, which is 90% Fe₂O₃ by mass, may be converted to magnetic microbeads using the methods described in Example 9 and Example 10.

### Example 16 (Prophetic): Preparation of PEG Surface Modification of Magnetic Beads

The material of Example 9 is dispersed in deionized water at 30% solids at natural pH to form a dispersion. An aqueous solution of α,ω-di-succinic acid polyethylene glycol (20,000 Da) is added in a sufficient quantity to yield a polyethylene glycol surface functionalized magnetic 88% Fe₂O₃ by mass following esterification reaction between the α,ω-di-succinic acid polyethylene glycol and surface silanols of the magnetic bead material of Example 9.

### Example 17 (Prophetic): Streptavidin Surface Modification of Magnetic Beads

1.0 kg of 3-aminoproyltriethoxysilane (CAS Number 919-30-2) are added to 5.0 kg of the material of Example 9 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol reaction product. The resultant material is further surface functionalized with streptavidin using glutaraldehyde as a coupling agent.

### Example 18 (Prophetic): Formation of a Dispersion of Single Domain Gamma Iron Oxide Magnetic Nanoparticles in a Polymer Matrix

0.446 kg of tetraethoxysilane (CAS Number 78-10-4), 0.542 kg of [3-(2,3-epoxypropoxy)-propyl]-trimethoxysilane (CAS Number 2530-83-8), and 0.05 kg of α,ω-silanol terminated poly(dimethylsiloxane) (CAS Number 70131-67-8) were added to 5.00 kg of the powder of Example 2 in a jacketed vacuum vessel under agitation at 25° C. under an inert nitrogen atmosphere. Under continued agitation, the temperature of the mixture was then raised to 110° C. and held constant for 1 hour. Under continued agitation while maintaining a temperature of 110° C., vacuum was applied to remove the ethanol and methanol reaction products and the powder is subsequently brought to a temperature of 50° C. The resultant material is coated with 10% by weight with a reactive variant of the crosspolymer compositions described in U.S. Pat. Nos. 9,139,737 and 10,590,278. This material may be further reacted with itself via catalytic homopolymerisation using either an anioic or cationic catalyst to yield a highly aggregated powder which may be converted to polymer coated magnetic microbeads using the methods described in Example 9 and Example 10. Alternately, this material may be further reacted with an appropriate polyphenol, amine, anhydride, or thiol and cured into large solid resin aggregates which may be converted to polymer coated magnetic microbeads using the methods described in Example 9 and Example 10.

### Example 19 (Prophetic): Formation of Complex Glass Magnetic Beads Using Top-Down Processing at High Throughput

A magnetic glass bead composition precursor suspension comprising 90% of the powder of Example 2 and 10% of the glass composition components of U.S. Pat. Pub. No. 2005/0266462 leading to a final composition of 70.67 mol % SiO₂, 14.33 mol % B₂O₃, 5.00 mol % Al₂O₃, 4.0 mol % K₂O and 2.00 mol % CaO is prepared as described in U.S. Pat. Pub. No. 2005/0266462. This precursor suspension is then spray dried to form glass beads having particle sizes of 25-100 microns, with 50 microns being a typical size. Spray drying is conducted using conventional high-throughput spray nozzle technologies resulting in significantly larger particles than disclosed in U.S. Pat. Pub. No. 2005/0266462. The use of this type of spray drying technology overcomes the significant product rate limitations that are well known when producing particles below 10 microns and does not require specialized sprayer designs. The resultant powder may be optionally sintered as described in U.S. Pat. Pub. No. 2005/0266462. The resultant powder is then transferred to large scale version of the orbital jet mill described in Examples 9 and 10 and processed under similar conditions to yield magnetic microbeads having averages sizes of 1-5 microns produced at industrial scale, multi-ton quantity overall throughput. The resultant magnetic beads may be optionally sintered as described in U.S. Pat. Pub. No. 2005/0266462, to control the shape of the beads.

### Example 20: Analysis of Magnetic Beads for the Separation and Purification of Nucleic Acids

The magnetic beads of Example 9 were dispersed in a 50% ethanol and 50% water solution, and they were compared against commercially available kits and platforms (hereafter referred to as the “Example 9 beads”). The first commercially available kit for testing was the MAGMAX™ Total Nucleic Acid Isolation Kit (THERMO FISHER SCIENTIFIC®, #AM1840). This kit was chosen for its universal applications (“RNA and genomic DNA from a variety of samples including viral, blood and bacterial samples”). The second kit was the MagNA Pure 24 Total NA Isolation Kit (ROCHE®, #07658036001). Similar to the MAGMAX™ kit, the MagNA Pure 24 Total NA Isolation kit was chosen for its broad utility to isolate nucleic acids (NA) from different sample materials and different sample volumes.

DNA Binding Assay

The DNA Binding and Recovery Properties of the Example 9 Beads and the Magnetic beads of the MAGMAX™ and MagNA kits were evaluated. To evaluate the magnetic beads, the beads were combined with a known concentration of DNA (initial concentrations). The magnetic beads bind to the nucleic acid, the magnetic beads are washed, and the DNA is eluted from the magnetic beads into solution. The eluted DNA is quantified to determine the percentage of the initial concentrations of DNA that are recovered. Initial concentrations were measured using the QUBIT™ dsDNA DNA BR Assay Kit (THERMO FISHER SCIENTIFIC®, #Q32850). These initial concentrations were created using serial dilutions. The DNA chosen for the assay was a commercially available solution of DNA typically used as a ladder in agarose gel electrophoresis applications: 1 kb DNA Ladder (New England Biolabs, #N3232L). This product was chosen because it contains a broad range of DNA lengths (500-10,000 base pairs) and has known concentrations of each length of DNA, which allows for determination of the varying sizes of eluted DNA through gel electrophoresis.

The Example 9 beads and MAGMAX™ beads were treated identically using the components provided in the MAGMAX™ Total Nucleic Acid Isolation Kit (specifically, binding, wash 1, wash 2, and elution buffers), with the exception that MAGMAX™ beads were treated with a “binding enhancer” solution prior to exposure to the DNA samples. The MSDS for this binding enhancer indicates it contains glycerol and proteinase K, though both concentrations are unlisted and the purpose of these additives is not stated. All reactions were scaled down in volume for compatibility with a 96-well plate assay and all measurements were performed in replicate. A total of 2 μL of either Example 9 beads or MAGMAX™ beads were used in each reaction. The ROCHE® MagNA kit was included later and was tested using the same protocol with MagNA buffers.

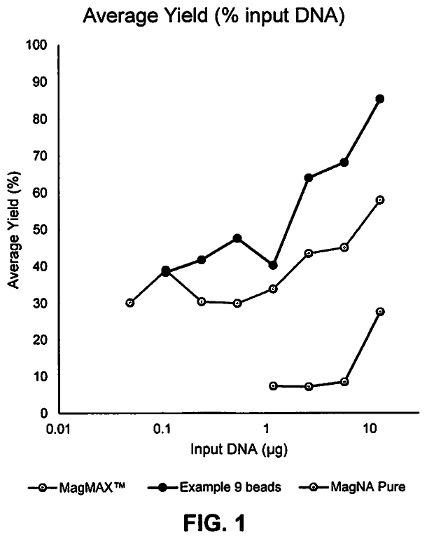

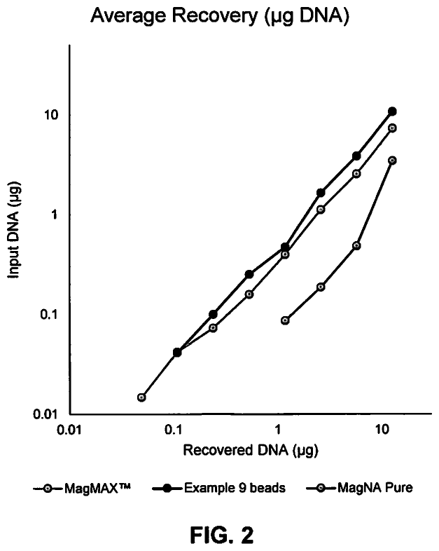

Following DNA binding, washing, and elution, samples were measured for DNA concentration using the QUBIT™ dsDNA DNA BR Assay Kit (THERMO FISHER SCIENTIFIC®, #Q32850) and QUBIT 4™ Fluorometer (THERMO FISHER SCIENTIFIC®, #Q33238). This assay was chosen due to its high sensitivity, high specificity for double stranded DNA, and broad working range of 100 pg/μL to 1,000 ng/μL. Input DNA concentrations spanned three orders of magnitude (10 ng to >10 μg) and were designed to saturate the MAGMAX™ beads. The average yields for each input DNA concentration are shown in Table 1 below. As shown in FIGS. 1 and 2, the Example 9 beads were observed to have higher recovery of input DNA (% yield) than MAGMAX™ beads at input DNA ≥0.108 μg. However, no binding was observed from the Example 9 beads at the lowest DNA inputs tested (0.022 μg and 0.049 μg). In contrast, MagMAX™ beads were able to recover DNA in duplicate at 0.049 μg and in a single replicate at 0.022 μg. Importantly, samples in which DNA was not detected using the QUBIT™ assay may still have DNA that can be recovered and amplified through methods like polymerase chain reaction (PCR), often used in diagnostic applications. Surprisingly, the MagNA kit performed the worst of all three beads tested and only showed purification at high input DNA concentrations.

TABLE 1
average yield

| | Average Yield (%) | | |
|---|---|---|---|
| Input DNA | | Example 9 | |
| (μg) | MAGMAX ™ | beads | MagNA Pure |
| 0 | — | — | — |
| 0.022 | — | — | — |
| 0.049 | 30.18 | — | — |
| 0.108 | 38.98 | 38.43 | — |
| 0.239 | 30.42 | 41.76 | — |
| 0.529 | 30.00 | 47.62 | — |
| 1.17 | 33.93 | 40.34 | 7.39 |
| 2.59 | 43.51 | 64.02 | 7.20 |
| 5.72 | 45.09 | 68.18 | 8.44 |
| 12.7 | 57.95 | 85.35 | 27.65 |

RNA Binding Assay

A commercially available ssRNA ladder (New England Biolabs, #N0362S) was chosen as an assay input. This ladder consists of 7 single stranded, linear RNA molecules ranging in size from 9000 base pairs down to 500.

Before purification, the ladder was denatured by heating in a heat block set to 65° C. for five minutes. The volume of beads used was 4 μL, after initial small-scale tests with 2 μL of beads showed unsatisfactory yields (data not shown). MAGMAX™ and the Example 9 beads were washed and eluted with the MAGMAX™ wash and elution buffers, while the MagNA Pure beads were washed and eluted with the MagNA Pure buffers.

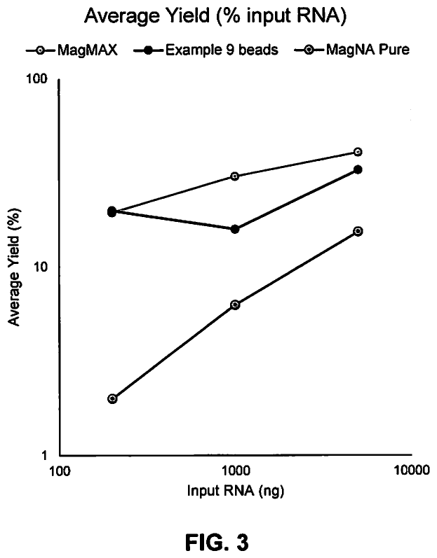

Initial RNA concentrations spanned >3 orders of magnitude, ranging from 5000 ng to 8 ng input. No bead functioned using 5.40 ng of input RNA, though a single outlier replicate of MAGMAX™ beads at the 8 ng input concentration showed a yield of almost 20 ng. This is higher than the input, so is likely a result of contamination at some point in the purification or quantification processes. MAGMAX™ and the Example 9 beads performed similarly at concentrations ≥200 ng, with all replicates recovering quantifiable RNA above this input level and only one replicate (MAGMAX™, 8 ng input) recovering QUBIT™ quantifiable RNA below it. This outlier is almost certainly an instrument error or sample contamination, as the amount recovered (662 ng) was much greater than the amount input. Both of these beads performed better than the MagNA Pure beads, which was only able to recover a detectable amount of RNA in a single replicate at 200 ng input, and at a low (6%) recovery. The average yield as a percentage of the initial input is shown in Table 2 below. The results are shown in FIGS. 3 and 4, which show the average yield as percentage of the input RNA and the average recovery in ng of RNA, respectively.

TABLE 2
Average yield
% Recovery

| | | |
|---|---|---|
| | Example 9 | |
| MAGMAX ™ | beads | MagNA Pure |

| Input | | Std. | | Std. | | Std. |
|---|---|---|---|---|---|---|
| (ng) | Mean | Dev. | Mean | Dev. | Mean | Dev. |
| 5000 | 40.60 | 0.69 | 32.60 | 1.25 | 15.26 | 3.36 |
| 1000 | 30.15 | 4.08 | 15.78 | 4.92 | 6.30 | 2.13 |
| 200 | 19.34 | 5.40 | 19.81 | 2.64 | 2.00 | — |
| 40 | — | — | — | — | — | — |
| 8 | * | — | — | — | — | — |

* A mean value much greater than input was likely the result of contamination and has been excluded.

DNA Binding—qPCR Assay

Analysis was carried out using quantitative PCR for analysis of bead performance due to its high sensitivity at low nucleic acid concentrations. Quantitative polymerase chain reaction (qPCR) is an umbrella term for a group of related assays which fluorescently track the accumulation of amplification products, either directly or indirectly, in real time. A TAQMAN™ based qPCR protocol was developed using a PCR amplified fragment of SARS-CoV-2 N gene as template and the N2 TAQMAN™ primers and probe (Integrated DNA Technologies, #10006713). 10 and 1 ng of amplified N-gene was used as input, as well a duplicate 0 ng input controls, and testing was performed in triplicate for the Example 9 beads and the MagNA Pure beads. Beads were washed and eluted almost identically to the protocol established with the QUBIT™ assay, with the exception that carrier nucleic acid was added to the lysis/binding buffer. One microliter of elution was added to 9 μL of TAQMAN™ Gene Expression Master Mix/N2 Primer Set and run in a ChaiBio Open qPCR set to 50 cycles of 50° C. annealing for 15 seconds, then 15 seconds of 68° C. extension.

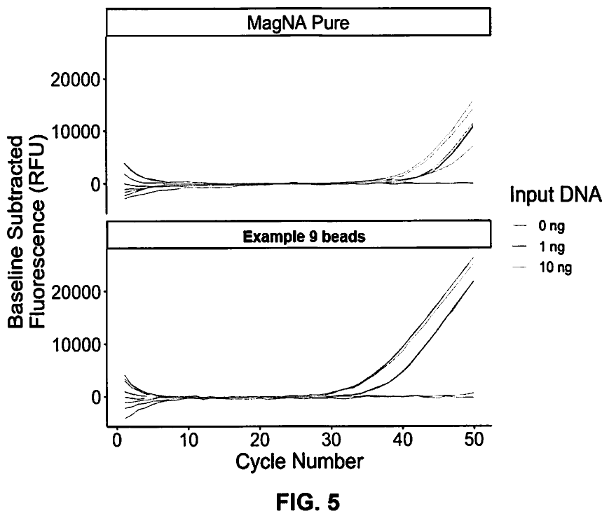

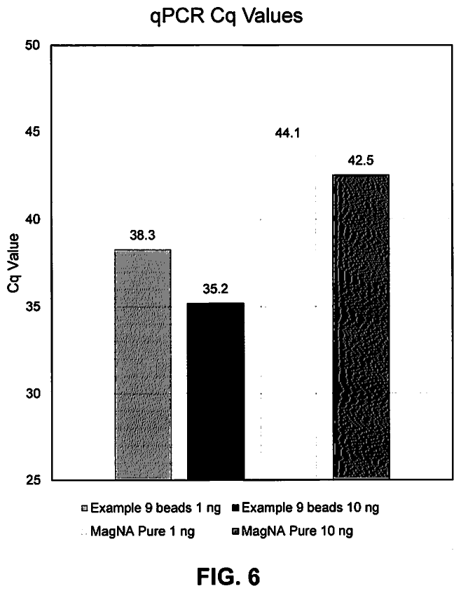

No N-gene was detected in any of the no-input negative controls, indicating that the carrier NA does not serve as a template for the primers. Similar to the QUBIT™ data, the Example 9 beads outperformed the MagNA Pure Beads, with consistently lower Cq values (the amplification cycle above which a curve is amplified above background). A lower Cq value indicates that the eluted solution had a higher concentration of DNA because a higher starting amount of DNA means that fewer cycles are needed to amplify the DNA to an amount above the background. Both sets of beads were able to reliably recover DNA down to 1 ng input, much lower than the lowest concentration with detectable output via QUBIT™. This data is shown in FIGS. 5 and 6.

RNA Binding-RT-qPCR Binding Assay

The qPCR described above was adapted into a reverse transcription qPCR (RT-qPCR) protocol. The forward primer used to amplify the SARS-CoV-2 N-gene fragment contained a T7 promoter, allowing for in vitro transcription of the above PCR product using T7 RNA polymerase (New England Biolabs, #E2040S). The result of this reaction is a linear, single stranded RNA fragment of the N-gene similar to what would be present in a patient sample, albeit unencapsulated.

Two input concentrations (10 ng and 50 ng RNA) were tested in triplicate and a 0 ng input negative control was tested in duplicate for Example 9 beads and MagNA Pure 24 beads. The same protocol for the samples prepared for RNA QUBIT™ assay was performed with the addition of carrier nucleic acid to the lysis/binding buffer. Four microliters of each bead were used due to previous experiments showing reduced binding capacity for RNA as compared to DNA. Samples were eluted in 30 μL of elution buffer from their beads' respective kits.

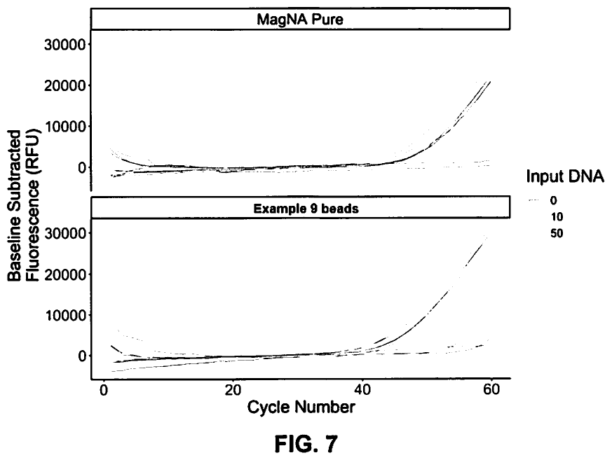

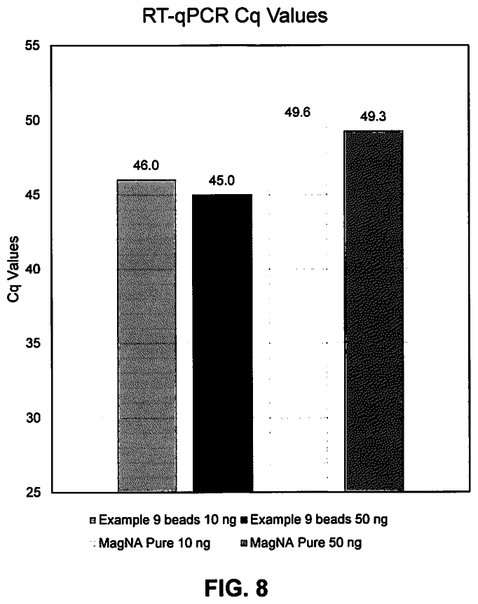

RNA from 5 μL of elution was reverse-transcribed into DNA using the LUNASCRIPT® RT SuperMix Kit (New England Biolabs, #E3010L), then 2 μL of reverse transcription mix was subjected to a scaled-up (20 μL as opposed to 10 μL) version of the qPCR assay used for DNA testing. TAQMAN™ Gene Expression Master Mix (Applied Biosystems, #4370048) and the N2 primer/probe set from Integrated DNA Technologies were used in the assay. Reactions were run in a ChaiBio Open qPCR, using the instruments software to calculate the cycle number at which fluorescence, and thus amplification, of a sample surpassed background. Assuming 100% PCR efficiency, a Cq value increase of 1 indicates that a sample has half as much DNA. Similar to previous results, Example 9 beads were better able to purify RNA than MagNA Pure beads. The Cq values obtained were generally higher than those acquired for DNA purification, indicating that RNA purification is less efficient than DNA purification for all beads tested (supported by the results of the QUBIT™ assays), the reverse transcriptase step is inefficient, or both. One of the measurements for the Example 9 beads for 0 ng inputs showed a Cq value of −55, suggesting contamination likely occurred during the microplate purification. Additionally, one of the Example 9 purifications had a Cq value of −55, likely due to an error in the purification process. Removing these outliers, the 10 ng input was recovered at Cq values of 46.0 for Example 9 beads and 49.6 for MagNA Pure, and the 50 ng input was recovered at Cq values of 45.0 for Example 9 beads and 49.3 for MagNA Pure. This data is shown in FIGS. 7 and 8.

### Example 21: Specific Surface Area of Magnetic Beads

Magnetic beads were prepared according to the process described in Example 8 with varying percentages of SiO₂ in the bead and tested after treatment at two different cross-linking temperatures, T1 and T2. In Table 3 below, T1 corresponds to a temperature of 85° C. and T2 corresponds to a temperature of 115° C. The specific surface area (SSA) of the magnetic beads were measured using the BET method. The specific surface areas of the beads increased as the percentage of SiO₂ increased. There was approximately a 20% decrease in the specific surface area when the cross-linking was performed under the higher temperature. The reduced surface area at T2 is presumably due to increased cross-linking at the higher temperature. There was no loss of saturation magnetization due to heating at either temperature. The sizes of these bead compositions may be modified to the desired bead size for the desired application.

TABLE 3

| | Saturation | | | % SSA Loss From |
|---|---|---|---|---|
| % SiO₂ in | Magnetization | T1 Processing | T2 Processing | Processing at 30° C. |
| Bead | (emu/g) | SSA (m²/g) | SSA (m²/g) | Increase |
| 0 | 72 | 32.0 | 32.0 | N/A |
| 5 | 68 | 56.4 | 44.2 | 21.6% |
| 10 | 65 | 86.7 | 69.0 | 20.4% |
| 15 | 61 | 112.1 | 90.6 | 19.2% |
| 20 | 58 | 166.3 | 132.4 | 20.4% |

### REFERENCES

1. Majetich, S. A. et al. “Magnetic nanoparticles” MRS Bulletin 38, pp. 899-903 (November 2013).
2. EP 0757106.
3. US 2005/0266462.
4. Shang, H., et al. “Synthesis and Characterization of Paramagnetic Microparticles through Emulsion-Templated Free Radical Polymerization” Langmuir 22, pp. 2516-2522 (2006).
5. U.S. Pat. No. 5,973,138
6. MagMAX™ Total Nucleic Acid Isolation Kit User Guide, Thermo Fisher Scientific, 2018.
7. U.S. Pat. No. 6,451,220
8. US Pat. Pub. 2003/0096987
9. US Pat. Pub. 2012/247150
10. US Pat. Pub. 2003/096987
11. Ogi, T. et al., “recent progress in nanoparticle dispersion using bead mill”, Kona Powder and Particle Journal, vol. 34, pp. 1-21 (2016).

1. Magnetic beads, comprising: (i) a plurality of magnetic nanoparticles, dispersed in (ii) a non-magnetic inorganic oxide matrix,
wherein the magnetic beads have an average particle size of 0.1 μm to 100 μm, the magnetic nanoparticles have an average particle size of 20 nm to 50 nm,
the non-magnetic inorganic oxide matrix contains neither Group I nor Group II elements, and does not contain boron, and
the magnetic beads contain at least 75% by weight of the plurality of magnetic nanoparticles, and retain a saturation magnetization of at least 75% of the bulk saturation of the magnetic nanoparticles, based on the weight of the magnetic beads.

2. Magnetic beads, comprising: (i) a plurality of magnetic nanoparticles, dispersed in (ii) a non-magnetic inorganic oxide matrix,
wherein the magnetic beads have an average particle size of 0.1 μm to 100 μm,
the magnetic nanoparticles have an average particle size of 20 nm to 50 nm,
the magnetic beads have a specific surface area of at least 40 m²/g, and
the magnetic beads contain at least 75% by weight of the plurality of magnetic nanoparticles, and retain a saturation magnetization of at least 75% of the bulk saturation of the magnetic nanoparticles, based on the weight of the magnetic beads.

3. The magnetic beads of claim 2, wherein the non-magnetic inorganic oxide matrix comprises SiO₂, Al₂O₃, TiO₂, or mixtures thereof.

4. Magnetic beads, comprising: (i) a plurality of magnetic nanoparticles, dispersed in (ii) a polymer matrix,
wherein the magnetic beads have an average particle size of 0.1 μm to 100 μm, and
the polymer matrix does not contain moieties of a PEG functionalized surfactant.

5. The magnetic beads of claim 4, wherein magnetic beads contain at least 75% by weight of the plurality of magnetic nanoparticles, and retain a saturation magnetization of at least 75% of the bulk saturation of the magnetic nanoparticles, based on the weight of the magnetic beads.

6. The magnetic beads of claim 2, wherein the beads do not comprise ion exchange resin.

7. The magnetic beads of claim 2, wherein the matrix comprises a siloxane, polyphenol, amine, anhydride, or thiol containing polymer.

8. The magnetic beads of claim 4, wherein the polymer matrix does not comprise polyphenol.

9. A method of making magnetic beads, comprising:
forming a solid dispersion comprising magnetic nanoparticles dispersed in a matrix; and
grinding the solid dispersion, to form magnetic beads having an average particle size of 0.1 μm to 100 μm.

10. The magnetic beads of claim 2, wherein the magnetic nanoparticles comprise gamma phase iron oxide and/or ferrite materials.

11. The magnetic beads or methods of claim 4, wherein the magnetic nanoparticles have an average particle size of 20 nm to 50 nm.

12. The magnetic beads of claim 2, further comprising a coating comprising PEG.

13. The magnetic beads of claim 2, further comprising a coating comprising a biological agent.

14. The magnetic beads of claim 2, having a saturation magnetization of at least 76% of the bulk saturation magnetization of the magnetic nanoparticles.

15. The magnetic beads of claim 2, having a saturation magnetization of 80% to 95% of the bulk saturation magnetization of the magnetic nanoparticles.

16. The magnetic beads of claim 1, wherein magnetic beads have a specific surface area of at least 40 m²/g.

17. A method of purifying nucleic acids, comprising:
providing the magnetic beads of claim 2,
reversibly binding the magnetic beads to nucleic acids,
washing the magnetic beads and nucleic acids, and
eluting the DNA fragments from the magnetic beads.

18. A method of reversibly binding at least one nucleic acid molecule, comprising:
(a) providing a dispersion of at least one magnetic bead of claim 2 in a solution;
(b) combining the dispersion with at least one nucleic acid molecule such that said at least one nucleic acid molecule is reversibly bound to said at least one magnetic bead;
eluting said at least one nucleic acid molecule from said at least one magnetic bead.

19. A kit comprising:
the magnetic beads of claim 2,
a binding solution,
a washing solution, and
an elution solution.

20. A polymerase chain reaction (PCR) kit, comprising:
the magnetic beads of claim 2, and
reagents for amplifying nucleic acids.
